(19) United States
(12) Patent Application Publication
(10) Pub. No.: US 2007/0066869 A1
(43) Pub. Date: Mar. 22, 2007

(54) Endoscopic assembly including cap and sheath

(76) Inventor: David Hoffman, Westborough, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/230,671

(22) Filed: Sep. 21, 2005

(51) Int. Cl. A61B 1/00

(52) U.S. Cl. 600/121; 600/114; 600/153

(57) ABSTRACT

An embodiment of the invention may include an endoscopic assembly. The endoscopic assembly may include an endoscope. The endoscopic assembly may also include an endoscopic cap and an endoscopic sheath each including a body defining a first channel accommodating the endoscope and a plurality of second channels disposed around the first channel. Each of the plurality of second channels may be configured to accommodate an endoscopic device therethrough.

### FIELD OF THE INVENTION

Embodiments of the invention include an endoscopic cap, an endoscopic sheath, and an endoscopic assembly including the endoscopic cap and the endoscopic sheath. Each of the endoscopic cap and the endoscopic sheath may include multiple channels.

### BACKGROUND OF THE INVENTION

Endoscopic methods are commonly used for diagnosis and/or treatment of the gastrointestinal tract. For example, there are several methods of treating esophageal cancer known as endoscopic mucosal resection. Endoscopic mucosal resection may include snaring and then excising sessile adenomas (i.e., tumors attached to a bodily surface) in the esophageal tract. If the adenoma is flat against the esophageal tract, thus making it difficult to snare and excise, one of several methods may be used to raise the flat adenoma so that it may be snared and excised. Such methods include, for example, using forceps, a vacuum, or injecting saline into the submucosa to raise the flat adenoma. Each of and methods may require a separate endoscopic mucosal resection device to be advanced separately down the esophageal tract to the site of the adenoma.

### SUMMARY OF THE INVENTION

An embodiment of the invention includes an endoscopic assembly. The endoscopic assembly includes a cap and an elongate sheath. Each of the cap and the elongate sheath define a first channel configured to accommodate an endoscope, and each of the cap and the elongate sheath define a plurality of second channels. Each of the plurality of second channels is configured to accommodate an endoscopic device therethrough.

Various embodiments of the invention may include one or more of the following aspects: the cap may define only one opening to the first channel defined by the cap; a portion of the first channel defined by the cap may be configured to form an interference fit with a distal end of an endoscope; the cap may be translucent; the endoscopic device may be one of a needle, grasper, snare, forceps, basket, wire-loop, and cutter; the plurality of second channels of the cap and the elongate sheath may be disposed around the first channel; an endoscope disposed in the first channel of the cap and the elongate sheath; the cap and elongate sheath may be integrally formed; the plurality of second channels of the cap may be aligned with the plurality of second channels of the elongate sheath; the first channel of the cap may be aligned with the first channel of the elongate sheath.

Another embodiment of the invention includes a method of performing and endoscopic procedure. The method includes providing an endoscopic assembly. The endoscopic assembly includes a cap and an elongate sheath. Each of the cap and the elongate sheath defines a first channel configured to accommodate an endoscope. Each of the cap and the elongate sheath defines a plurality of second channels. Each of the plurality of second channels is configured to accommodate an endoscopic device therethrough. The method further includes advancing the endoscopic assembly through a body lumen to a treatment site, advancing a first endoscopic device through one of the plurality of second channels, treating the treatment site using the first endoscopic device, and retracting the first endoscopic device through the one of the plurality of second channels.

Various embodiments of the invention may include one or more of the following aspects: the endoscopic assembly based on a view of the treatment site obtained from the endoscope; the cap may define only one opening to the first channel defined by the cap; a portion of the first channel defined by the cap may be configured to form an interference fit with a distal end of an endoscope; the cap may be translucent; the endoscopic device may be one of a needle, grasper, snare, forceps, basket, wire-loop, and cutter; advancing a second endoscopic device through another of the plurality of second channels; treating the treatment site using the second endoscopic device; retracting the second endoscopic device through the another of the plurality of second channels; the endoscopic procedure may be an endoscopic mucosal resection procedure; the first endoscopic device may be capable of injecting fluid into tissue and the second endoscopic device may be capable of removing tissue; the first endoscopic device may be an injection needle and the second endoscopic device may be a snare; the treating using the first endoscopic device and the treating using the second endoscopic device may be performed substantially simultaneously; the second endoscopic device may be advanced while the first endoscopic device is disposed in the one of the plurality of second channels; the plurality of second channels may be disposed around the first channel; the cap and elongate sheath may be integrally formed; the plurality of second channels of the cap may be aligned with the plurality of second channels of the elongate sheath; the first channel of the cap may be aligned with the first channel of the elongate sheath.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate several embodiments of the invention and together with the description, serve to explain the principles of the invention.

### BRIEF DESCRIPTION OF THE DRAWINGS

FIG. 1 is a schematic view of an endoscopic assembly according to an embodiment of the invention;

FIG. 2 is a schematic view of an endoscopic cap of the endoscopic assembly of FIG. 1;

FIG. 3 is a schematic view of an endoscopic sheath of the endoscopic assembly of FIG. 1;

FIG. 4 is a schematic view of the endoscopic cap of FIG. 2 accommodating an endoscopic device;

FIG. 5 is a schematic view of an endoscopic assembly according to another embodiment of the invention;

FIG. 6A is a schematic view of an endoscopic cap and an endoscopic sheath according to a further embodiment of the invention;

FIG. 6B is a schematic view of an endoscopic cap and an endoscopic sheath according to a still another embodiment of the invention;

FIG. 6C is a schematic view of an endoscopic cap and an endoscopic sheath according to a still further embodiment of the invention; and

FIG. 7 is a schematic view of an endoscopic assembly disposed in a body lumen proximate a treatment site according to yet another embodiment of the invention.

### DESCRIPTION OF THE EMBODIMENTS

Reference will now be made in detail to the present embodiments of the invention, examples of which are illustrated in the accompanying drawings. Wherever possible, the same reference numbers will be used throughout the drawings to refer to the same or like parts.

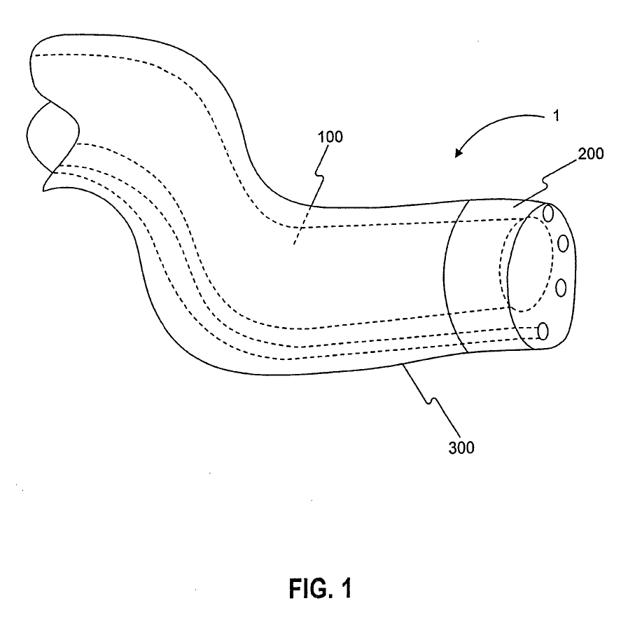

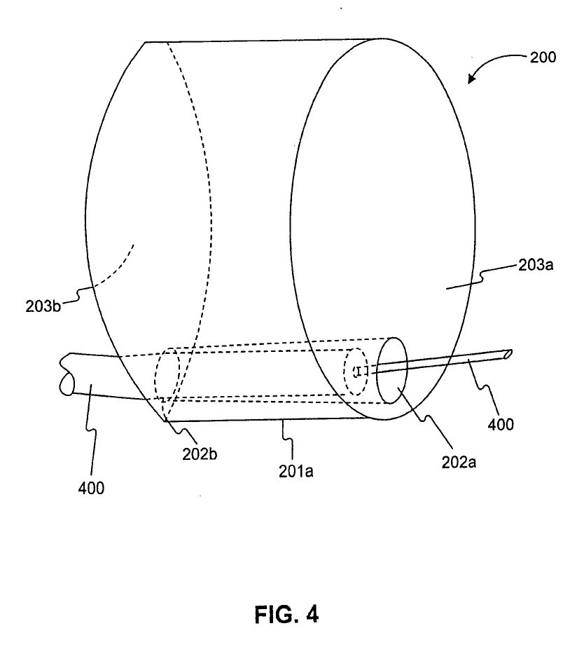

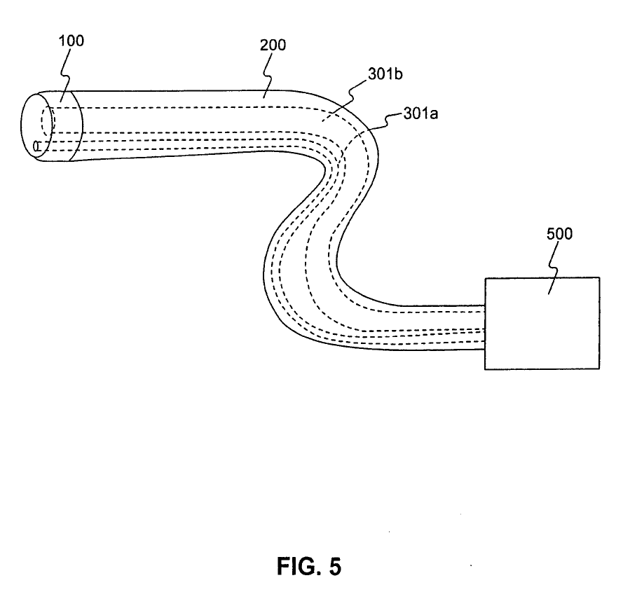

FIG. 1 depicts an endoscopic assembly 1 according to an embodiment of the invention. Endoscopic assembly 1 may include one or more of endoscopic device 100, endoscopic cap 200, and endoscopic sheath 300. As depicted in FIGS. 4 and 5, assembly 1 may also include one or more endoscopic devices 400 and a handle 500.

Endoscopic device 100 may be any suitable endoscopic and/or medical device, for example, an endoscope, laprascope, ureteroscope, hysteroscope, and/or a flexible bronchoscope. Endoscopic device 100 may be configured to allow a user to view a body lumen via a distal end of the endoscopic device. Endoscopic device 100 may also include one or more components of a variceal banding system that may be used, for example, in a endoscopic variceal banding ligation procedure. Such a procedure may include placing small elastic bands around varices in the distal 5 cm of the esophagus. Varices may be suctioned into the banding device and then the bands may be released around the base of the varix by pulling a trip wire via a biopsy channel.

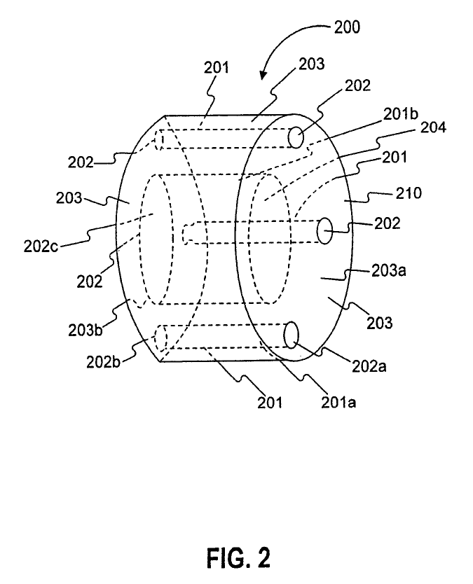

FIG. 2 depicts an exemplary embodiment of endoscopic cap 200. Endoscopic cap 200 may be made out of any suitable biocompatible material, for example, rubber or plastic. Endoscopic cap 200 may be made using any suitable method, for example, injection molding or machining.

Endoscopic cap 200 may include a body 210 defining a plurality of channels 201 with openings 202 on one or more surfaces 203 of endoscopic cap 200. For example, a first channel 201a may extend through endoscopic cap 200, and may have a first opening 202a on a first surface 203a and a second opening 202b on a second surface 203b. First channel 201a may be substantially parallel to a longitudinal axis of endoscopic cap 200. First channel 201a may be configured to accommodate an endoscopic device therethrough, for example, an injection needle 400 as shown in FIG. 4 or any other endoscopic device, such as, for example, a needle, grasper, snare, forceps, basket, wire-loop, cutter, dilation balloon, stent, scissors, stapler, suture mechanism, clip, endo-loop, guidewire, microendoscope, knife, needle knife, sensor device, guide catheter, and/or other surgical instruments. First channel 201a may have a substantially consistent cross-sectional area for its entire length. Endoscopic cap 200 may have a plurality of channels 201 similar to first channel 201a disposed about different portions of endoscopic cap 200. The plurality of channels 201 may be substantially parallel to each other.

In another example, a second channel 201b may extend partially through endoscopic cap 200, and may have only a first opening 202c on second surface 203b. Second channel 201b may be substantially parallel to a longitudinal axis of endoscopic cap 200. Second channel 201b may be configured to accommodate an endoscopic device therein, for example, an endoscope as shown in FIG. 1. Channel 201b therefore may be larger than channels 201a. Second channel 201b may have a substantially consistent cross-sectional area for its entire length. Second channel 201b may have a cross-sectional area greater than first channel 201a. Second channel 201b may have an interior surface 204. Interior surface 204 may have a shape that is similar to a cross-section of second channel 201b. Interior surface 204 may have a surface area that is similar to an area of a cross-sectional portion of second channel 201b. Interior surface 204 may be substantially parallel to second surface 203b. Second channel 201b may be substantially coaxial with a longitudinal axis of endoscopic cap 200. Second channel 201b may be configured to form an interference fit around a distal end of endoscopic device 100.

At least a portion of endoscopic cap 200 may be translucent and/or made of a translucent material. For example, at least a portion of the endoscopic cap 200 between interior surface 204 and first surface 203a may be translucent such that when distal end of endoscope 100 is disposed in second channel 201b, endoscope 100 may be able to provide a visual image to the user of the area past surface 203a, for example, so that the user may be able to see a treatment site when endoscopic assembly 1 is disposed in a body lumen. Such a translucent region may have a cross-sectional area substantially the same as the cross-sectional area of second channel 201b.

In various embodiments, one or more aspects of endoscopic cap 200 may be altered. Different portions of endoscopic cap 200 may be made of different materials. For example, a portion between interior surface 204 and first surface 203a may be translucent while the rest of endoscopic cap 200 may be opaque. Channel 201 may take any path through endoscopic cap 200, for example, channel 201 may include a bend. Openings 202 may be disposed on any surface 203. Channel 201 may include two or more branches and/or may include two or more openings 202. Channel 201 may vary in cross-sectional area and/or shape, for example, along its length. Channel 201 and/or opening 202 may have any cross-sectional shape. Surface 203 may have any geometrical shape and/or contour. Channels 201 of cap 200 may differ from one another, for example, differing cross-sectional shapes and/or areas, and at least some of which may take different paths through body 210. The translucent region in endoscopic cap 200 may have a cross-sectional area greater or less than the cross-sectional area of second channel 201b. Channels 201a may be disposed adjacent to and/or around channel 201b in any suitable configuration.

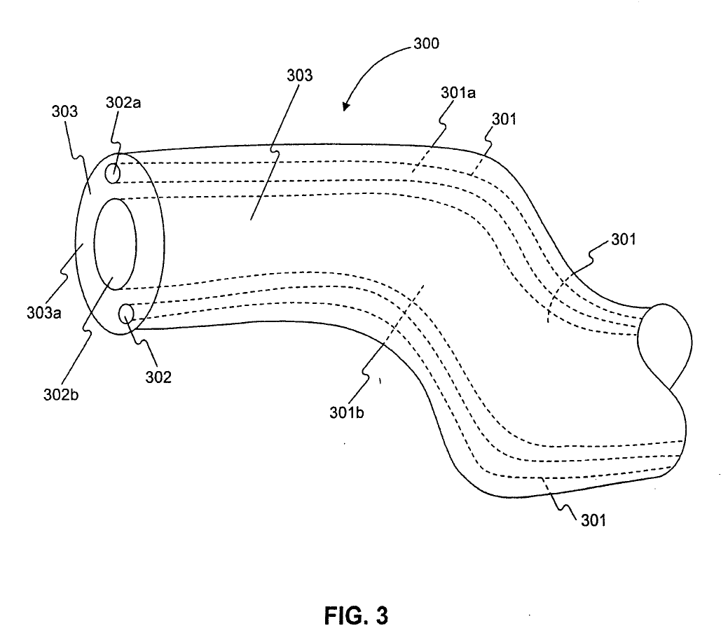

FIG. 3 depicts an exemplary embodiment of endoscopic sheath 300. Endoscopic sheath 300 may have a substantially elongate configuration, for example, so that it may be advanced through a tortuous body lumen. Endoscopic sheath 300 may be made out of any suitable biocompatible material, for example, rubber or plastic. Endoscopic sheath 300 may be made using any suitable method, for example, by extrusion. At least a portion of endoscopic sheath 300 may be translucent and/or made of a translucent material. Endoscopic sheath 300 may be configured as an elongate member.

Endoscopic sheath 300 may include a plurality of channels 301 with one or more openings 302 on one or more surfaces 303 of endoscopic sheath 300. For example, a first channel 301a may extend through endoscopic sheath 300, and may have a first opening 302a on a first surface 303a. First channel 301a may extend through endoscopic sheath 300 to handle 500, for example, as shown in FIG. 5. First channel 301a may be substantially parallel to a longitudinal axis of endoscopic sheath 300. First channel 301a may be configured to accommodate an endoscopic device 400 therethrough, for example, an injection needle as shown in FIG. 4 or any other endoscopic device, such as, for example, a needle, grasper, snare, forceps, basket, wire-loop, cutter, dilation balloon, stent, scissors, stapler, suture mechanism, clip, endo-loop, guidewire, microendoscope, knife, needle knife, sensor device, guide catheter, and/or other surgical instruments. First channel 301a may have a substantially consistent cross-sectional area for its entire length. Endoscopic sheath 300 may have a plurality of channels similar to first channel 301a disposed about different portions of endoscopic sheath 300.

In another example, a second channel 301b may extend partially or fully through endoscopic sheath 300, and may have a first opening 302b on first surface 303a. Second channel 301b may extend through endoscopic sheath 300 to handle 500, for example, as shown in FIG. 5. Second channel 301b may be substantially parallel to a longitudinal axis of endoscopic sheath 300. Second channel 301b may be configured to accommodate an endoscopic device therethrough, for example, an endoscopic device 100 as shown in FIG. 1, and particularly an endoscope. Channel 301b therefore may be larger than channels 301a. Second channel 301b may have a substantially consistent cross-sectional area for its entire length. Second channel 301b may have a cross-sectional area greater than first channel 301a. Second channel 301b may be substantially coaxial with a longitudinal axis of endoscopic sheath 300.

In various embodiments, one or more aspects of endoscopic sheath 300 may be altered. Different portions of endoscopic sheath 300 may be made of different materials. Sheath 300 may have areas reinforced by braiding, coils, wires, coextrusions, or the like. Sheath 300 may have areas of variable stiffness, for example, to provide flexibility along at least a portion of the length of sheath 300. Sheath 300 may include one or more coatings (e.g., lubricious coatings) on at least a portion of its inside surface and/or outside surface, for example, to aid in the implementation of the sheath 300 in the body. Channel 301 may take any path through endoscopic sheath 300, for example, channel 301 may have portions that are not parallel to a longitudinal axis of endoscopic sheath 300. Openings 302 may be disposed on any surface 303. Channel 301 may include two or more branches and/or may include two or more openings 302. Channel 301 may vary in cross-sectional area and/or shape, for example, along its length. Channel 301 and/or opening 302 may have any cross-sectional shape. Surface 303 may have any geometrical shape and/or contour. Channels 301 of sheath 300 may differ from one another, for example, differing cross-sectional shapes and/or areas. Channels 301a may be disposed adjacent to and/or around channel 301b in any suitable configuration. Sheath 300 and/or channels 301a may have different shapes and/or contours, for example, to permit insertion of specifically shaped and/or contoured instruments.

Assembly 1 may include a handle 500, for example, as shown in FIG. 5. Handle 500 may be any suitable handle disposed at a proximal end of sheath 300. During use of assembly 1 (e.g., while cap 200 and/or sheath 300 is being advanced into a body lumen), handle 500 may remain outside the body lumen and/or body. Handle 500 may be configured to allow endoscope 100 and/or endoscopic device 400 to be advanced into channels 201, 301. For example, handle 500 may have access ports corresponding and/or aligned with channels 301 so as to allow endoscope 100 and/or endoscope device 400 to be advanced into channels 301 via those access ports. Handle 500 may be configured to allow insertion of a plurality of instruments at the same time.

Cap 200 and sheath 300 may be formed as an integral assembly or may be manufactured separately and joined together in any suitable manner. For example, surface 203b of cap 200 may be configured to be connected to endoscopic sheath 300 and/or surface 303a may be configured to be connected to endoscopic cap 200. In an embodiment, surface 203b may be substantially flat so as to be attached to surface 303a of endoscopic sheath 300 via an adhesive, and surface 303a may be substantially flat so as to be attached to surface 203b of endoscopic cap 200 via an adhesive.

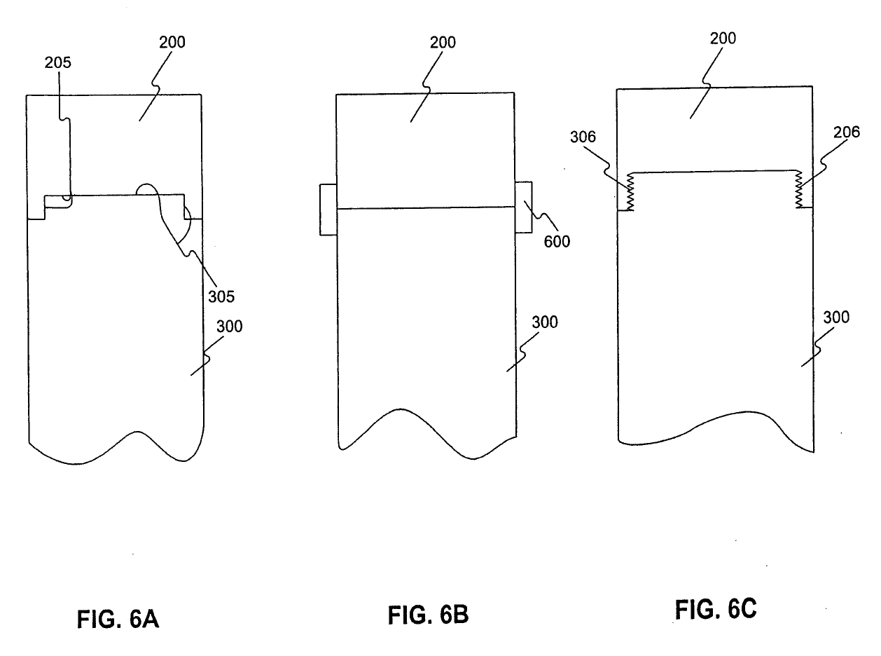

In another example, as shown in FIG. 6A, endoscopic cap 200 may include a recessed portion 205 configured to accommodate a protruding portion 305 of endoscopic sheath 300. Recessed portion 205 and protruding portion 305 may be configured to form an interference fit with each other. For example, a cross-sectional area of recessed portion 205 may be slightly smaller than a cross-sectional area of protruding portion 305. However, in alternate embodiments, endoscopic sheath 300 may include a recessed portion and endoscopic cap 200 may include a protruding portion.

In another example, as shown in FIG. 6B, endoscopic assembly 1 may include a collar 600 configured to hold endoscopic cap 200 and endoscopic sheath 300 together. Collar 600 may form an interference fit with one or more of endoscopic cap 200 and endoscopic sheath 300. For example, collar 600 may be annular and define a cross-sectional cavity or hole therein that is slightly smaller than a cross-sectional area of endoscopic cap 200 and/or endoscopic sheath 300. Collar 600 may also or alternatively be located inside cap 200 and may allow for a good fit of cap 200 around endoscope 300.

In a further example, as shown in FIG. 6C, an internally threaded portion 206 of endoscopic cap 200 may be configured to be screwed onto an externally threaded portion 306 of endoscopic sheath 300. However, in alternate embodiments, endoscopic sheath 300 may be configured to be screwed onto endoscopic cap 200. Endoscopic cap 200 and endoscopic sheath 300 may be threaded, for example, such that channels 201 align with channels 301 when endoscopic cap 200 is screwed onto endoscopic sheath 300 or vice versa. Other methods of attaching endoscopic cap 200 to endoscopic sheath 300 may include clipping, snapping, anchoring, and/or ratcheting one of endoscopic cap 200 to endoscopic sheath 300. Endoscopic cap 200 and endoscopic sheath 300 may also be integrally formed such that both may be placed over endoscope 100 substantially simultaneously. In alternative embodiments, devices may be used to secure endoscopic 200 to endoscopic sheath 300, for example, clips, snaps, anchors, ratchets, adhesives, and/or velcro tape.

Endoscopic cap 200 and/or endoscopic sheath 300 may include means for aligning channels 201 relative to channels 301 such that they form one substantially continuous channel. For example, endoscopic cap 200 and endoscopic sheath 300 may be integrally formed or may be threaded as set forth in FIG. 6C. In another example, one or more of endoscopic cap 200 and/or endoscopic sheath 300 may include one or more markings (e.g., on an exterior surface of one or more of endoscopic cap 200 and/or endoscopic sheath 300) that may be used to align channels 201 relative to channels 301. In a further example, a portion of one of endoscopic cap 200 and endoscopic sheath 300 defining channels 201 and channels 301, respectively, may be configured to move longitudinally relative to the one of endoscopic cap 200 and endoscopic sheath 300 and/or may be configured to be placed in a recess in the other of endoscopic cap 200 and endoscopic sheath 300. Thus, channels 201 of endoscopic cap 200 may extend from an end of endoscopic cap 200 and may be configured to placed into endoscopic sheath 300 such that endoscopic sheath 300 surrounds at least a portion of channels 201. In yet another example, one or more of endoscopic cap 200 and endoscopic sheath 300 may include tabs that are configured to align and/or mate with slots on the other of endosopic cap 200 and endoscopic sheath 300. In some embodiments, both endoscopic cap 200 and endoscopic sheath 300 may include tabs and slots. In a yet further example, endoscopic cap 200 and endoscopic sheath 300 may have contoured mating surfaces that correspond to each other such that when endoscopic cap 200 and endoscopic sheath 300 are properly joined such that the contoured mating surfaces are aligned, channels 201 and 301 may be aligned.

Corresponding channels 201, 301 may have substantially the same cross-sectional area and/or shape. For example, channels 201a, 301a may have substantially the same cross-sectional area and/or shape. In another example, channels 201b, 301b may have substantially the same cross-sectional area and/or shape.

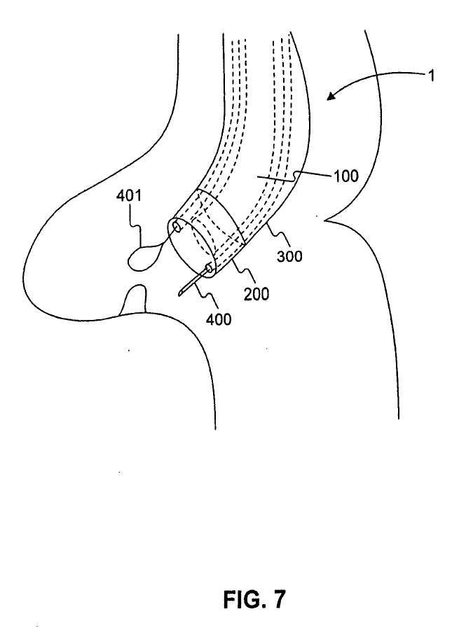

An embodiment of the invention may include a method of using endoscopic assembly 1, for example, as shown in FIG. 7. The method may include providing endoscopic assembly 1, which may include one or more of endoscopic device 100, endoscopic cap 200, endoscopic sheath 300, one or more endoscopic devices 400, and handle 500. As described, endoscopic cap 200 may be connected to endoscopic sheath 300 in any suitable method, such as those described herein.

Endoscopic device 100, such as an endoscope with visualiziation capability, may be advanced through sheath 300. Sheath 300 may be placed over endoscopic device 100 such that endoscopic device 100 is disposed in channel 301b. A distal end of endoscopic device 100 may protrude from a distal end of sheath 300, for example, through opening 302b of surface 303a.

Endoscopic cap 200 may be placed over the distal end of endoscopic device 100, for example, in channel 201b through opening 202c. The distal end of endoscopic device 100 may contact surface 204 of channel 201b. The surfaces of channel 201b may form an interference fit around the distal end of endoscopic device 100 to aid in retaining the distal end of device 100 in channel 201b.

Endoscopic assembly 1 may be advanced through a body lumen, for example, an esophageal tract, to a treatment site. Assembly 1 may be advanced together as a unit. Alternatively, sheath 300 and cap 200 first may be advanced to the site, and device 100 later inserted within sheath 300 to the site. Endoscopic device 400 then may be advanced through channel 301a, opening 302a, opening 202b, channel 201a, and opening 202a into the esophageal tract. Endoscopic device 400 may then be manipulated (e.g., using a handle separate from handle 500) to perform a step in a medical procedure. For example, in a mucosal resection procedure, a needle 400 may be used to inject saline and raise a flat adenoma. As another example, a snare 401 may be used to excise a raised adenoma. The endoscope 100 may be used to visualize these procedures. Once the step has been completed, endoscopic device 400 (or 401) may be advanced out of the esophageal tract, out of opening 202a, channel 201a, opening 202b, opening 302a, and channel 301a.

Another endoscopic device 400 may be advanced through, disposed in, and/or retracted from the same or a different channel 201, 301 of endoscopic assembly 1. One or more endoscopic devices 400 may be advanced through, disposed in, and/or retracted from one or more channels 201, 301 of endoscopic assembly 1 at substantially the same time, for example, to perform multiple steps of a medical procedure substantially simultaneously or at least relatively close together in time.

In various embodiments, assembly 1 may only include a sheath 300 and not cap 200. Thus, only sheath 300 may be advanced into the body lumen to the treatment site, and endoscope 100 and/or endoscopic device 400 may only be advanced and/or retracted through channels 301 of endoscopic sheath 300.

There are many benefits and advantages to embodiments of the invention. For example, a medical procedure, such as a mucosal resection, may take less time because multiple endoscopic devices may not need to be continuously advanced and/or retracted from a body lumen. Instead, multiple endoscopic devices may be advanced and/or retracted from the body lumen substantially simultaneously. In another example, multiple endoscopic devices may be used to treat a treatment site at substantially the same time. In a further example, embodiments of the invention may reduce the chance that the multiple endoscopic devices will interfere, for example, by having a dedicated channel 201, 301 for each endoscopic device.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An endoscopic assembly, including:
a cap and an elongate sheath, each of the cap and the elongate sheath defining a first channel configured to accommodate an endoscope, and each of the cap and the elongate sheath defining a plurality of second channels,
wherein each of the plurality of second channels is configured to accommodate an endoscopic device therethrough.

2. The endoscopic assembly of claim 1, wherein the cap defines only one opening to the first channel defined by the cap.

3. The endoscopic assembly of claim 1, wherein a portion of the first channel defined by the cap is configured to form an interference fit with a distal end of an endoscope.

4. The endoscopic assembly of claim 1, wherein the cap is translucent.

5. The endoscopic assembly of claim 1, wherein the endoscopic device is one of a needle, grasper, snare, forceps, basket, wire-loop, and cutter.

6. The endoscopic assembly of claim 1, wherein the plurality of second channels of the cap and the elongate sheath are disposed around the first channel.

7. The endoscopic assembly of claim 1, further comprising an endoscope disposed in the first channel of the cap and the elongate sheath.

8. The endoscopic assembly of claim 1, wherein the cap and elongate sheath are integrally formed.

9. The endoscopic assembly of claim 1, wherein the plurality of second channels of the cap are aligned with the plurality of second channels of the elongate sheath.

10. The endoscopic assembly of claim 1, wherein the first channel of the cap is aligned with the first channel of the elongate sheath.

11. A method of performing and endoscopic procedure, comprising:
providing an endoscopic assembly, including:
a cap and an elongate sheath, each of the cap and the elongate sheath defining a first channel configured to accommodate an endoscope, and each of the cap and the elongate sheath defining a plurality of second channels,
wherein each of the plurality of second channels is configured to accommodate an endoscopic device therethrough,
advancing the endoscopic assembly through a body lumen to a treatment site;
advancing a first endoscopic device through one of the plurality of second channels;
treating the treatment site using the first endoscopic device; and
retracting the first endoscopic device through the one of the plurality of second channels.

12. The method of claim 11, further comprising orienting the endoscopic assembly based on a view of the treatment site obtained from the endoscope.

13. The method of claim 11, wherein the cap defines only one opening to the first channel defined by the cap.

14. The method of claim 11, wherein a portion of the first channel defined by the cap is configured to form an interference fit with a distal end of an endoscope.

15. The method of claim 11, wherein the cap is translucent.

16. The method of claim 11, wherein the endoscopic device is one of a needle, grasper, snare, forceps, basket, wire-loop, and cutter.

17. The method of claim 11, further comprising advancing a second endoscopic device through another of the plurality of second channels;
treating the treatment site using the second endoscopic device; and
retracting the second endoscopic device through the another of the plurality of second channels.

18. The method of claim 17, wherein the endoscopic procedure is an endoscopic mucosal resection procedure, the first endoscopic device is capable of injecting fluid into tissue, and the second endoscopic device is capable of removing tissue.

19. The method of claim 18, wherein the first endoscopic device is an injection needle and the second endoscopic device is a snare.

20. The method of claim 17, wherein the treating using the first endoscopic device and the treating using the second endoscopic device are performed substantially simultaneously.

21. The method of claim 17, wherein the second endoscopic device is advanced while the first endoscopic device is disposed in the one of the plurality of second channels.

22. The method of claim 11, wherein the plurality of second channels are disposed around the first channel.

23. The method of claim 11, wherein the cap and elongate sheath are integrally formed.

24. The method of claim 11, wherein the plurality of second channels of the cap are aligned with the plurality of second channels of the elongate sheath.

25. The method of claim 11, wherein the first channel of the cap is aligned with the first channel of the elongate sheath.
